(19) United States
(12) Patent Application Publication
(10) Pub. No.: US 2024/0277483 A1
(43) Pub. Date: Aug. 22, 2024

(54) KNEE PROSTHESIS FOR ERROR CORRECTION AND USE METHOD THEREOF

(71) Applicants: THE THIRD HOSPITAL OF CHANGSHA, Changsha City (CN); BEIJING LIDAKANG TECHNOLOGY CO., LTD., Beijing (CN)

(72) Inventors: Tangyou LIU, Changsha City (CN); Xian YU, Changsha City (CN); Shuqin MA, Changsha City (CN); Qing LEI, Changsha City (CN)

(73) Assignees: THE THIRD HOSPITAL OF CHANGSHA, Changsha City (CN); BEIJING LIDAKANG TECHNOLOGY CO., LTD., Beijing (CN)

(21) Appl. No.: 18/431,130

(22) Filed: Feb. 2, 2024

(30) Foreign Application Priority Data
Feb. 21, 2023 (CN) 202310140415.8

(51) Int. Cl. A61F 2/38; A61B 90/00; A61F 2/30

(52) U.S. Cl. CPC A61F 2/389; A61F 2/30734; A61B 2090/3762; A61F 2002/305; A61F 2002/3069; A61F 2002/3082

(57) ABSTRACT

Disclosed is a knee prosthesis for error correction and a use method thereof, belonging to the technical field of knee prostheses. A knee prosthesis for error correction includes a tibial insert and a tibia tray. The tibial insert is arranged on the tibia tray, and includes a first spacer, a second spacer and a supporting spacer. The first spacer and the second spacer are respectively located on both sides of the supporting spacer and spliced to form a whole spacer. The whole spacer is provided with an anti-falling piece, and the whole spacer is clamped with a tibia tray platform. The supporting spacer is provided with a connecting piece, and the supporting spacer is respectively connected to the first spacer and the second spacer through the connecting piece. According to the present disclosure, the problem of imbalanced spacing between femur and tibia after surgery caused by improper osteotomy is improved.

### CROSS-REFERENCE TO RELATED APPLICATION

This patent application claims the benefit and priority of Chinese Patent Application No. 202310140415.8, filed with the China National Intellectual Property Administration on Feb. 21, 2023, the disclosure of which is incorporated by reference herein in its entirety as part of the present application.

### TECHNICAL FIELD

The present disclosure relates to the technical field of knee prostheses, and in particular to a knee prosthesis for error correction and a use method thereof.

### BACKGROUND

Knee arthroplasty is a surgery to relieve knee joint pain and correct knee joint deformity using an artificial prosthesis, thus improving the knee joint. Late knee joint pain can be effectively eradicated, and the quality of life of the patient can be greatly improved.

General knee prosthesis includes a femoral condyle prosthesis, a tibial insert and a tibial tray. The femoral condyle is fixedly connected to distal femur, the tibial tray is fixedly connected to an upper end of tibia, and the tibial insert is arranged between the femoral condyle prosthesis and the tibial tray, so as to replace the diseased knee joint and achieve normal walking and knee bending functions.

Osteotomy of all parts in knee arthroplasty is the core of knee arthroplasty. If there is no correct reference standard for the positioning of composite osteotomy of distal femur and osteotomy of proximal tibia in the surgery, and the relationship between normal medial and lateral condyles of femur and the anatomy of proximal tibia is unclear, improper osteotomy may be caused. When using traditional tibial insert products, lower extremity alignment varus and valgus, large posterior inclination of tibia and imbalanced flexion-extension gap of the knee joint will occur after surgery.

### SUMMARY

In order to improve the problem of imbalanced spacing between femur and tibia after surgery caused by improper osteotomy, a knee prosthesis for error correction and a use method thereof are provided by the present disclosure.

In a first aspect, a knee prosthesis for error correction is provided, which employs the following technical solutions:

A knee prosthesis for error correction includes a tibial insert and a tibial tray. The tibial insert is arranged on the tibial tray. The tibial tray includes a tibial tray platform and a tibial intramedullary nail, and the tibial tray platform is connected to the tibial intramedullary nail.

The tibial insert includes a first spacer, a second spacer, and a supporting spacer. The first spacer and the second spacer are respectively located on both sides of the supporting spacer and spliced into a whole spacer. The whole spacer is provided with an anti-falling piece, and the whole spacer is clamped with the tibial tray platform through the anti-falling piece.

The supporting spacer is provided with a connecting piece, the supporting spacer is respectively connected to the first spacer and the second spacer through the connecting piece.

By employing the above technical solution, the whole spacer is clamped with the tibial tray platform through the anti-falling piece, which improves the connection stability of the tibial insert and the tibial tray, and is also convenient for replacement. The whole spacer is divided into three parts: a first spacer, a second spacer, and a supporting spacer. When the knee joint of a patient is inclined to one side, the spacer on this side can be replaced to correct the inclined knee joint, there is no need to perform osteotomy again, the growth and development of the knee joint are facilitated, and the pain of the patient is reduced.

Alternatively, an edge of the tibial tray platform bulges to form a flange, and whole spacer is clamped into the flange.

By employing the above technical solution, the flange is formed by an edge of the tibial tray platform. When the whole spacer is clamped into the flange, the flange can play a role in limiting the whole spacer, thus reducing the slippage of the whole spacer on the tibial tray platform.

Alternatively, the anti-falling piece includes a clamping strip, and the clamping strip is arranged on the whole spacer. The flange is provided with a first clamping groove, and the clamping strip is clamped into the first clamping groove.

By employing the above technical solution, the clamping strip can be clamped with the first clamping groove, which not only can improve the connection stability of the whole spacer and the tibial tray platform, but also can facilitate the whole spacer to be clamped to and separated from the tibial tray platform, thus facilitating the replacement of the whole spacer.

Alternatively, the whole spacer is provided with an annular groove, and the flange is clamped into the annular groove. The anti-falling piece includes a clamping strip, and the clamping strip is arranged on a side wall of the annular groove, and the clamping strip is clamped into the first clamping groove.

By employing the above technical solution, the clamping strip is clamped into the first clamping groove, and thus the connection stability of the whole spacer and the tibial tray platform is improved.

Alternatively, the connecting piece includes clamping strips, and the clamping strips are respectively arranged on both sides of the supporting spacer. The first spacer and the second spacer are both provided with second clamping grooves, and the clamping strips are respectively clamped with the second clamping grooves.

By employing the above technical solution, the clamping strip is clamped with the second clamping groove, thus connecting the first spacer, the second spacer and the supporting spacer to form the whole spacer. Force in horizontal and vertical directions can be transferred between all parts of the whole spacer.

Alternatively, the tibial tray platform is provided with a clamping block, the supporting spacer is provided with a holding groove, and the clamping block is clamped into the holding groove.

By employing the above technical solution, the clamping block is clamped with the holding groove to facilitate the mounting and dismounting of the supporting spacer and the tibial tray platform as well as improve the connection stability of the whole spacer and the tibial tray platform.

Alternatively, the flange is provided with a receding groove, and the receding groove is located on one side, away from the clamping block, of the tibial tray platform.

By employing the technical solution above, in the process of clamping/separating the supporting spacer to/from the clamping block, the receding groove can provide a space for movement, thus facilitating the mounting and dismounting of the supporting spacer and the clamping block.

Alternatively, a cross-sectional area of the clamping block gradually increases in a direction from the supporting spacer to the tibial tray platform.

By employing the technical solution above, the cross-sectional area of the clamping block gradually decreases in a direction from top to bottom, thus facilitating the first spacer, the second spacer and the supporting spacer to transfer and share the force in the horizontal direction.

Alternatively, the cross-sectional area of the clamping block gradually decreases in a direction from the clamping block to the receding groove.

By employing the technical solution above, the first spacer and the second spacer can prevent the supporting spacer from slipping out, thus improving the connection stability of the whole spacer.

In a second aspect, a method for using the knee prosthesis for error correction above is provided, including the following steps:

S1: performing CT scanning on a patient to determine an inclination direction of a knee joint of the patient;

S2: performing surgery on the knee joint of the patient, and actually measuring a knee joint spacing and knee joint pressure; and

S3: selecting and replacing a first spacer or a second spacer.

By employing the technical solution above, the appropriate first spacer or second spacer can be replaced for the patient, thus reducing the opening of surgical trauma and improve the imbalance of knee joint gap.

In conclusion, the present disclosure has at least one of the following beneficial effects:

1. The first spacer and the second spacer are respectively located on both sides of the supporting spacer. When the knee joint of the patient is inclined, the imbalanced knee joint can be corrected by replacing the first spacer or the second spacer.

2. The clamping strip enables the first spacer, the second spacer and the supporting spacer to transfer the power, so as to improve the connection stability.

3. The clamping strip is clamped into the first clamping groove, so as to facilitate the mounting and dismounting of the whole spacer and the tibial tray platform.

### BRIEF DESCRIPTION OF THE DRAWINGS

FIG. 1 is an overall schematic diagram of a knee prosthesis for error correction according to Embodiment 1 of the present disclosure;

FIG. 2 is an exploded schematic diagram of a knee prosthesis for error correction according to Embodiment 1 of the present disclosure;

FIG. 3 is a schematic diagram showing a clamping strip according to Embodiment 1 of the present disclosure;

FIG. 4 is an exploded schematic diagram of a knee prosthesis for error correction according to Embodiment 2 of the present disclosure;

FIG. 5 is a schematic diagram showing a clamping strip according to Embodiment 2 of the present disclosure.

In the drawings: 1—tibial insert; 11—first spacer; 12—second spacer; 13—supporting spacer; 131—holding groove; 14—supporting post; 15—anti-falling piece; 151—clamping strip; 16—connecting piece; 161—clamping strip; 17—second clamping groove; 18—annular groove; 2—tibial tray; 21—tibial tray platform; 22—tibial intramedullary nail; 23—wing plate; 24—flange; 241—first clamping groove; 25—clamping block; 26—embedding cavity; 27—receding groove.

### DETAILED DESCRIPTION OF THE EMBODIMENTS

The present disclosure is further described below with reference to accompanying drawings 1-5.

### Embodiment 1

A knee prosthesis for error correction is disclosed by Embodiment 1 of the present disclosure.

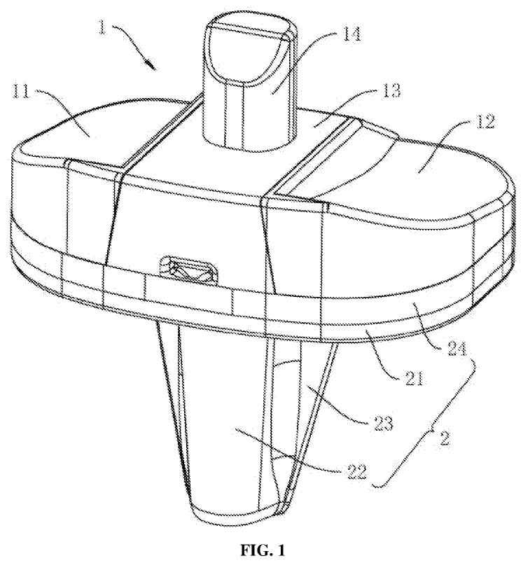

Referring to FIG. 1, the knee prosthesis for error correction includes a tibial insert 1 and a tibial tray 2. The tibial insert 1 is clamped to the tibial tray 2, and a femoral condyle prosthesis (not shown in figure) is arranged on the tibial insert 1. A diseased knee joint is subjected to osteotomy, such that the femoral condyle prosthesis is fixedly connected to distal femur, and a lower end of the tibial tray 2 is inserted into tibia, thus achieving the replacement of the diseased knee joint.

Referring to FIG. 1, the tibial tray 2 includes a tibial tray platform 21 and a tibial intramedullary nail 22, the tibial intramedullary nail 22 is fixedly connected to a bottom wall of the tibial tray platform 21, and the tibial intramedullary nail 22 is perpendicular to the tibial tray platform 21. The tibial intramedullary nail 22 is inserted into one end, close to the knee joint, of the tibia, thus fixing the tibial tray 2 to the tibia. The tibial tray 2 further includes a wing plate 23, one end of the wing plate 23 is fixedly connected to a bottom wall of the tibial tray 2, and the other end of the wing plate 23 is fixedly connected to a side wall of the tibial intramedullary nail 22. In this embodiment, there are two wing plates 23, which are arranged at two opposite sides of the tibial intramedullary nail 22, thus supporting the tibial tray platform 21.

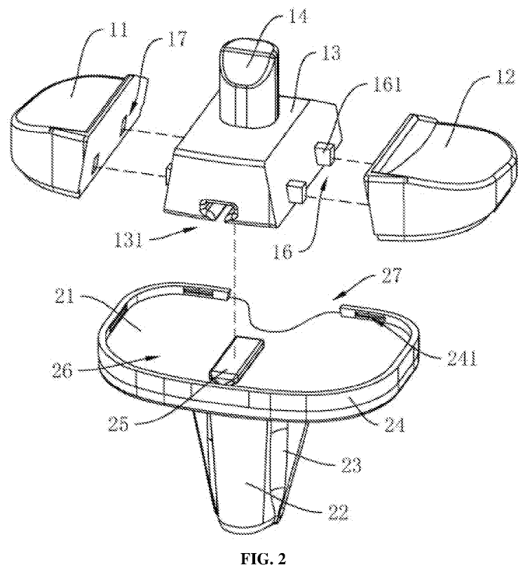

Referring to FIG. 2, an edge of the tibial tray platform 21 bulges upwards to form a flange 24, the flange 24 and the tibial tray platform 21 form an embedded cavity 26 for embedding the tibial insert 1, and a side wall of the flange 24 plays a role in limiting the tibial insert 1, thus improving the connection stability of the tibial tray 2 and the tibial insert 1.

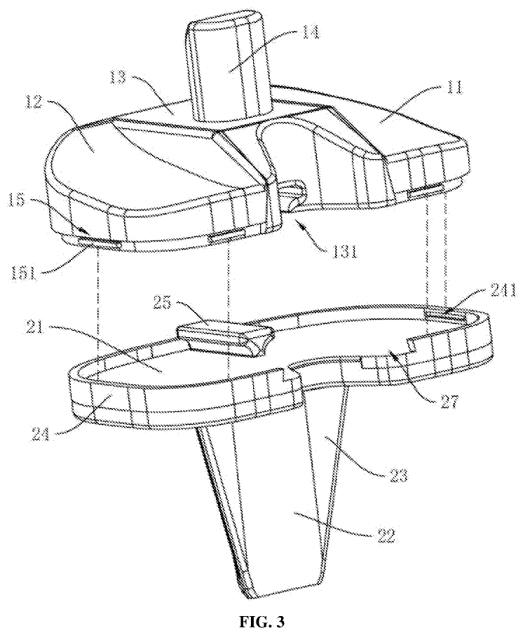

Referring to FIG. 3, a clamping block 25 is fixedly connected to the tibial tray platform 21 and extends from a front edge of the tibial tray platform 21 to a middle position. In a direction from top to bottom, the cross-sectional area of the upper end of the clamping block 25 is greater than that of the lower end. One side, close to the tibial tray platform 21, of the supporting spacer 13 is provided with a holding groove 131, the clamping block 25 is in fit with the holding groove 131, so as to clamp the supporting spacer 13 to the tibial tray platform 21. A receding groove 27 is reserved on a front edge position, away from the tibial tray platform 21, of the flange 24, thus clamping the supporting spacer 13 into the clamping block 25 conveniently.

Referring to FIG. 2, the tibial insert 1 includes a first spacer 11, a second spacer 12, and a supporting spacer 13. The first spacer 11 and the second spacer 12 are symmetrically arranged, and the first spacer 11 and the second spacer 12 are respectively located on both sides of the supporting spacer 13. A side wall of the supporting spacer 13 in a length direction is inclined. Specifically, the cross-sectional area of the supporting spacer 13 gradually increases in a direction from top to bottom, and gradually decreases in a direction from front to back. The first spacer 11, the second spacer 12 and the supporting spacer 13 are combined to form a whole spacer, and the whole spacer can be embedded into the embedding cavity 26.

Referring to FIG. 2, the connecting piece 16 includes clamping strips 161, the clamping strips 161 are integrally formed on both sides of the supporting spacer 13 in a length direction. The first spacer 11 and the second spacer 12 are provided with second clamping grooves 17 at corresponding potions, and the clamping strips 16 on both sides of the supporting spacer 13 can be clamped into the second clamping grooves 17, respectively.

When the whole spacer is embedded into the embedding cavity 26, as the side wall of the supporting spacer 13 is inclined, and the clamping strips 161 are respectively clamped into the second clamping grooves 17, the first spacer 11, the second spacer 12 and the supporting spacer 13 can transfer the force in a horizontal direction to one another. Meanwhile, the first spacer 11 and the second spacer 12 can prevent the supporting spacer 13 from slipping out from the receding groove 27, thus improving the connection stability.

Referring to FIG. 2 and FIG. 3, the anti-falling piece 15 includes clamping strips 151, and the clamping strips 151 are integrally formed on a peripheral side wall of the first spacer 11 and the second spacer 12. An inner wall of the flange 24 is provided with first clamping grooves 241, the clamping strips 151 are provided with a certain elasticity, and can be clamped into the first clamping grooves 241, thus clamping the first spacer 11 and the second spacer 12 to the tibial tray platform 21.

Referring to FIG. 3, a supporting post 14 is fixedly connected to one side, away from the tibial tray platform 21, of the supporting spacer 13, the supporting post 14 can be connected to the femoral condyle prosthesis above without affecting the normal knee bending action of the human body.

When the knee joint of the patient is laterally inclined due to excessive lateral osteotomy, the patient is subjected to CT scanning, and then subjected to a surgery to determine an inclination spacing of the patient, and a thicker first spacer 11 is replaced for the patient to correct the inclined knee joint. When the knee joint of the patient is medially inclined due to excessive medial osteotomy, a thicker second spacer 12 is replaced for the patient to correct the knee joint. When the knee joint is large in posterior inclination due to excessive posterior osteotomy, the first space 11 and the second spacer 12 need to be replaced at the same time.

Specifically, when the knee joint of the patients is inclined medially or laterally, a bottom wall of each of the first spacer 11 and the second spacer 12 before replacement is a flat plane, and a bottom wall of the first spacer 11 or the second spacer 12 after replacement is inclined. The cross-sectional area gradually decreases from a direction where the first spacer 11 or the second spacer 12 approaches the supporting spacer 13, that is, one side, away from the supporting spacer 13, of the first spacer 11 or the second spacer 12 is higher in height, and one side, near the supporting spacer 13, of the first spacer 11 or the second spacer 12 is lower. The first spacer 11 or second spacer 12 in an inclined state can offset the inclined knee joint spacing, thus correcting the knee joint.

When the knee joint of the patient is inclined posteriorly, the first spacer 11 and the second spacer 12 need to be replaced at the same time. In a direction from the receding groove 27 to the clamping block 25, the cross-sectional area of the first spacer 11 or the second spacer 12 gradually decreases, thus correcting the posteriorly inclined knee joint.

It is worth noting that multiple first spacers 11 and second spacers 12 with different thickness specifications are reserved, and all the first spacers 11 and second spacers 12 with different thicknesses can be clamped with the tibial tray platform 21 to facilitate the replacement during surgery.

In Embodiment 1, the implementation principle of the knee prosthesis for error correction is as follows: the first spacer 11, the second spacer 12 and the supporting spacer 13 are spliced to form a whole spacer, the whole spacer is clamped into the holding groove 131 through the clamping block 25, and the clamping strip 151 is clamped into the first clamping groove 241, so as to achieve the clamping of the whole spacer and the tibial tray platform 21. When the knee joint of a patient is inclined, the corresponding first spacer 11 or second spacer 12 is replaced, so as to correct the knee joint and reduce surgical trauma.

### Embodiment 2

A knee prosthesis for error correction is disclosed by Embodiment 2 of the present disclosure.

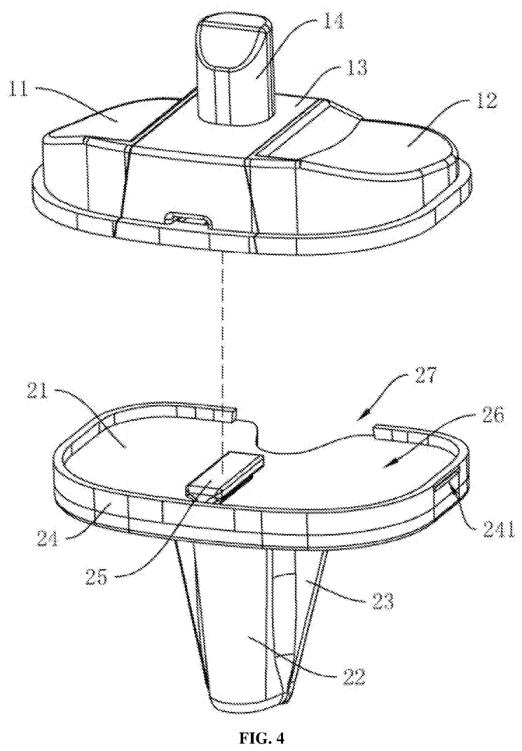

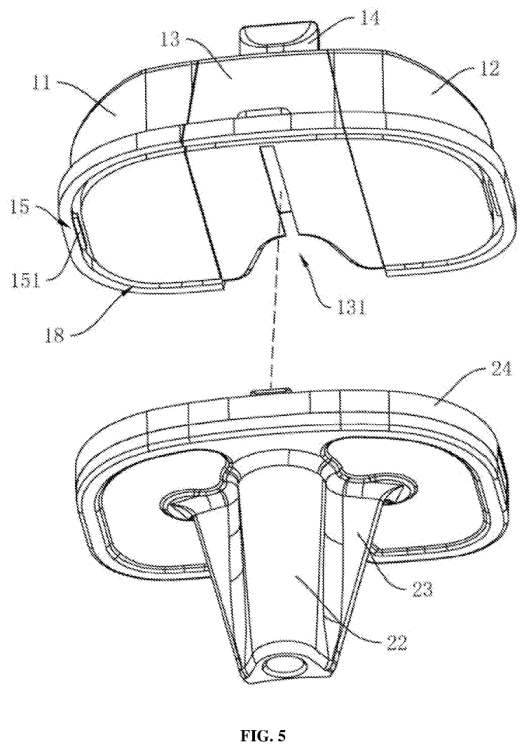

Referring to FIG. 4 and FIG. 5, the difference between Embodiment 2 and Embodiment 1 is that the area of the whole spacer is slightly greater than that of the tibial tray platform 21; one side, close to the tibial tray platform 21, of the tibial insert 1 is provided with an annular groove 18, the flange 24 is clamped into the annular groove 18, and the whole spacer can be attached to the tibial tray platform 21.

Referring to FIG. 4 and FIG. 5, the clamping strip 151 is fixedly connected to a side wall of the annular groove 18, the flange 24 is provided with a first clamping groove 241, and the clamping strip 151 can be clamped into the first clamping groove 241. Specifically, the annular groove 18 has two side walls. In this embodiment, the clamping strip 151 is located on an outer side wall of the annular groove 18. In some embodiments of the present disclosure, the clamping strip 151 is located on an inner side wall of the annular groove 18.

### Embodiment 3

A method for using the knee prosthesis for error correction in Embodiment 1 or Embodiment 2 is disclosed by Embodiment 3 of the present disclosure, including the following steps:

S1. A patient is subjected to CT scanning to determine an inclination direction of a knee joint of the patient.

S2. The knee joint of the patient of is subjected to surgery, and a knee joint spacing and knee joint pressure are actually measured.

S3. An appropriate first spacer 11 or second spacer 12 is selected to replace.

The above are the preferred embodiments of the present disclosure, and the scope of protection of the present disclosure is not limited accordingly. Therefore, all equivalent changes made according to the structure, shape and principle of the present disclosure should be included in the scope of protection of the present disclosure.

1. A knee prosthesis for error correction, comprising a tibial insert and a tibial tray, wherein the tibial insert is arranged on the tibial tray; the tibial tray comprises a tibial tray platform and a tibial intramedullary nail, and the tibial tray platform is connected to the tibial intramedullary nail;
the tibial insert comprises a first spacer, a second spacer, and a supporting spacer; the first spacer and the second spacer are respectively located on both sides of the supporting spacer and spliced into a whole spacer; the whole spacer is provided with an anti-falling piece, and the whole spacer is clamped with the tibial tray platform through the anti-falling piece;
the supporting spacer is provided with a connecting piece, the supporting spacer is respectively connected to the first spacer and the second spacer through the connecting piece.

2. The knee prosthesis for error correction according to claim 1, wherein an edge of the tibial tray platform bulges to form a flange, and whole spacer is clamped into the flange.

3. The knee prosthesis for error correction according to claim 2, wherein the anti-falling piece comprises a clamping strip, and the clamping strip is arranged on the whole spacer; the flange is provided with a first clamping groove, and the clamping strip is clamped into the first clamping groove.

4. The knee prosthesis for error correction according to claim 2, wherein the whole spacer is provided with an annular groove, and the flange is clamped into the annular groove; the anti-falling piece comprises a clamping strip, and the clamping strip is arranged on a side wall of the annular groove; and the flange is provided with a first clamping groove, and the clamping strip is clamped into the first clamping groove.

5. The knee prosthesis for error correction according to claim 1, wherein the connecting piece comprises clamping strips, and the clamping strips are respectively arranged on both sides of the supporting spacer; the first spacer and the second spacer are both provided with second clamping grooves, and the clamping strips are respectively clamped with the second clamping grooves.

6. The knee prosthesis for error correction according to claim 2, wherein the tibial tray platform is provided with a clamping block, the supporting spacer is provided with a holding groove, and the clamping block is clamped into the holding groove.

7. The knee prosthesis for error correction according to claim 6, wherein the flange is provided with a receding groove, and the receding groove is located on one side, away from the clamping block, of the tibial tray platform.

8. The knee prosthesis for error correction according to claim 6, wherein a cross-sectional area of the clamping block gradually increases in a direction from the supporting spacer to the tibial tray platform.

9. The knee prosthesis for error correction according to claim 7, wherein the cross-sectional area of the clamping block gradually decreases in a direction from the clamping block to the receding groove.

10. A method for using the knee prosthesis for error correction according to claim 1, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

11. The method for using the knee prosthesis for error correction according to claim 2, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

12. The method for using the knee prosthesis for error correction according to claim 3, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

13. The method for using the knee prosthesis for error correction according to claim 4, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

14. The method for using the knee prosthesis for error correction according to claim 5, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

15. The method for using the knee prosthesis for error correction according to claim 6, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

16. The method for using the knee prosthesis for error correction according to claim 7, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

17. The method for using the knee prosthesis for error correction according to claim 8, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.

18. The method for using the knee prosthesis for error correction according to claim 9, comprising the following steps:
S1: performing CT scanning on a patient to determine an inclination direction of a knee joint of the patient;
S2: performing surgery on the knee joint of the patient, and actually measuring a knee joint spacing and knee joint pressure; and
S3: selecting and replacing a first spacer or a second spacer.
